(19) United States
(12) Patent Application Publication
(10) Pub. No.: US 2025/0366776 A1
(43) Pub. Date: Dec. 4, 2025

(54) Home Based Portable Piezoelectric Detection Device And System

(71) Applicant: United Arab Emirates University, Al Ain (AE)

(72) Inventor: Mahmoud Al Ahmad, Al Ain (AE)

(21) Appl. No.: 18/732,158

(22) Filed: Jun. 3, 2024

(51) Int. Cl. A61B 5/05; A61B 5/00

(52) U.S. Cl. CPC A61B 5/4312; A61B 5/0285; A61B 5/6843; A61B 5/742; A61B 2562/0247; A61B 2562/04

(57) ABSTRACT

A detection system for breast cancer includes a detection device; and a mobile application for displaying information. The device includes an array of piezoelectric sensors, a plastically deformable layer, a ridged layer, and an electrically coupled system for processing voltage signals from the sensors which are arranged a predetermined distance from one another and imbedded in the layer. The layer is located beneath layer and has a hemispherical shape. In use, the sensors produce a voltage in response to a force applied thereto, when the sensors are compressed between breast tissue and the layer. The electrically coupled system receives signals from the sensors which are transformed mathematically into pressure readings. Mathematical modelling and/or graphical data representation is then applied to the pressure readings to produce a graphic output which is compared to a reference library.

### FIELD OF INVENTION

This invention relates to a home based portable piezoelectric detection device, system and method.

### BACKGROUND TO INVENTION

The market interest for early detection of breast cancer is substantial and growing. The global breast cancer diagnostics market, valued at USD 4.3 billion in 2022, is anticipated to expand at a compound annual growth rate (CAGR) of 7.4% from 2023 to 2030. This growth is propelled by the increasing incidence of breast cancer worldwide and the recognition of the critical role that early detection plays in managing the disease effectively.

Traditionally, women undergo breast cancer screening on a regular basis such as annually or as part of routine and regular checkups. Typically, such screenings are performed in a clinical setting whereby women undergo physical examinations and mammograms. One disadvantage of traditional screening methods is that it is inconvenient, intrusive, and unpleasant. Therefore, some women avoid routine screening. A need therefore exists for less intrusive, less unpleasant and more convenient screening methods.

Another disadvantage of traditional screening methods is that women who avoid regular screenings, for the abovementioned reasons, or for cost reasons, fail to catch cancer at an early stage, which greatly impacts the success rate of treatment. Accordingly, a need exists for early detection cancer screening devices which are private, cost effective and convenient and which do not involve the disadvantages mentioned above.

U.S. Pat. No. 10,076,247B2-discloses use of piezoelectric fingers (PEFS) to determine whether artificial tumours embedded in artificial tissue samples have a rough or branchy interfacial surface, a potential indicator of invasive malignant cancer such as malignant breast cancer. This was determined by measuring the elastic modulus (E), shear modulus (G) and determining the G/E ratio for the artificial tissues. An image, graphical or numerical representation of the spatial distribution of the elastic modulus was produced. U.S. Pat. No. 10,076,247B2 further teaches applying a voltage for actuation of the device.

The arrangement disclosed in U.S. Pat. No. 10,076,247B2 is disadvantageous as it requires a power source. U.S. Pat. No. 10,076,247B2 further teaches the use of at least four stainless steel cantilever structure which form the fingers. This arrangement is overly complex and cumbersome, and not ideally suited for comfort when pressing the fingers against breast tissue. Also this is not a very portable arrangement.

### SUMMARY OF INVENTION

According to a first aspect of the invention, there is provided a detection method including:
providing at least two piezoelectric sensors spaced a predetermined distance apart from one another;
providing a plastically deformable material and locating said at least two piezoelectric sensors on the plastically deformable material;
providing a rigid material adjacent said at least two piezoelectric sensors; compressing said at least two piezoelectric sensors between the rigid material and a material to be measured, thereby to produce a voltage signal in said at least two piezoelectric sensors; and transforming the voltage signals from said at least two piezoelectric sensors into a measurement of a desired property of the material being measured.

In a particular embodiment, the material to be measured may be in the form of living tissue. The living tissue may be in the form of breast tissue.

In a particular embodiment, the voltage signals from said at least two piezoelectric sensors may be transformed into a measurement of viscosity of blood flowing in the breast tissue beneath the sensors.

In another embodiment, the voltage signals from said at least two piezoelectric sensors into may be transformed into a measurement of reaction forces of the tissue. In yet another embodiment, the voltage signals from said at least two piezoelectric sensors into may be transformed into a measurement of radius of a blood vessel of the breast tissue. In still another embodiment, the voltage signals from said at least two piezoelectric sensors into may be transformed into a measurement of velocity of blood flow in a blood vessel of the breast tissue.

Providing at least two piezoelectric sensors may comprise providing a plurality of sensors. More specifically, providing at least two piezoelectric sensors may comprise providing an array of sensors arranged a predetermined distance apart from one another. In a particular embodiment, said predetermined distance may be a uniform distance such that the array of sensors are spaced an equal distance apart from one another.

According to a second aspect of the invention, there is provided a detection device including:
at least two piezoelectric sensors spaced a predetermined distance apart from one another;
a plastically deformable material layer;
said at least two piezoelectric sensors being located adjacent the plastically deformable material layer; and
a rigid layer located adjacent the plastically deformable material layer, in an arrangement wherein, in use, said at least two sensors each produce a voltage signal in response to a force applied to said at least two sensors when the piezoelectric sensors are compressed, in use, between a material to be measured and the rigid layer.

In a particular embodiment, the detection device may include a plurality of sensors. More specifically, the plurality of sensors may comprise an array of sensors arranged a predetermined distance apart from one another. In a particular embodiment, said predetermined distance may be a uniform distance such that the array of sensors are spaced an equal distance apart from one another.

In a particular embodiment, the material to be measured may be in the form of living tissue. The living tissue may be in the form of breast tissue.

In a particular embodiment, the device may be operable for transforming the voltage signal from said at least two sensors into pressure readings for detecting changes in pressure between adjacent sensors in the array of sensors.

In a particular embodiment, the device may be operable to construct a map of the pressure distribution for specific areas of the breast. As such, the device may be operable to compare the map with one or more maps obtained from a reference library. Said reference library may include one or more maps of pressure distributions obtained from healthy individuals using the device.

In a particular embodiment, said one or more sensors may be at least partially imbedded in the plastically deformable layer.

The detection device may be operable for detecting material properties of the breast tissue, as will be explained in more detail hereinbelow. In a particular embodiment, the material properties may be one or more of the following: a mechanical stress of the tissue, a viscosity of the tissue, a radius of a blood vessel of the tissue, a reaction force of the tissue.

In a particular embodiment, the rigid layer may have a hemispherical shape. The device may include an electrically coupled system including electric circuits connected to the sensors and configured to process the signals. The electric circuits may include an interface circuit for receiving and amplifying the signals received from each one of said one or more sensors. The electric circuit may further include a microcontroller for receiving amplified signals from the interface circuit. The electric circuits may further include a digital signal processor (DSP) for processing complex computations on digital signals received from the microcontroller. As such, the DSP may be operable for analysing patterns or detecting anomalies. The electric circuits may further include a communication module for communication with a portable mobile device, such as, for example, a cellular phone or smartphone. The electric circuits may further include a storage module.

According to another aspect of the invention there is provided a portable piezoelectric detection system, the system including:
a device as described and defined hereinabove; and
a mobile application which can be installed, in use, on the smartphone or cellular phone and which is operable for displaying information received from the device. In use, said information may include visual data provided for the user.

### BRIEF DESCRIPTION OF DRAWINGS

Further features of the invention are described hereinafter by way of a non-limiting example of the invention, with reference to and as illustrated in the accompanying schematic drawings. In the drawings:

FIG. 1 shows a flowchart of a detection method, in accordance with a first aspect of the invention;

FIG. 2 shows a schematic view of the piezoelectric detection device, in accordance with one aspect of the invention, shown in use, for detecting breast cancer; and a detection system, in accordance with another aspect of the invention;

FIG. 3 shows a sectional view of a head of the piezoelectric detection device of FIG. 2;

FIG. 4 shows a side view of an end portion of the head of the device of FIG. 3;

FIG. 5 shows an end view of the head of the device of FIG. 4;

FIG. 6 shows a flowchart of the workings of the piezoelectric detection device of FIG. 2; and the detection system of FIG. 2;

FIG. 7A shows a schematic depiction of various tumours of different sizes;

FIG. 7B shows a schematic depiction of various tumours of different sizes of FIG. 7A, shown imbedded in a gelatine-based breast model;

FIG. 8A shows a depiction of a measured pressure distribution of the sensors of FIG. 2;

FIG. 8B shows a depiction of a pressure observed/calculated between the sensors of FIG. 8A;

FIG. 9 shows the sensors of the piezoelectric detection device of FIG. 2, located above a blood vessel in a breast of a subject;

FIG. 10 shows a graphic representation of results from mathematic modelling and/or Multiphysics® simulations and/or the Coventorware® software.

### DETAILED DESCRIPTION OF THE DRAWINGS

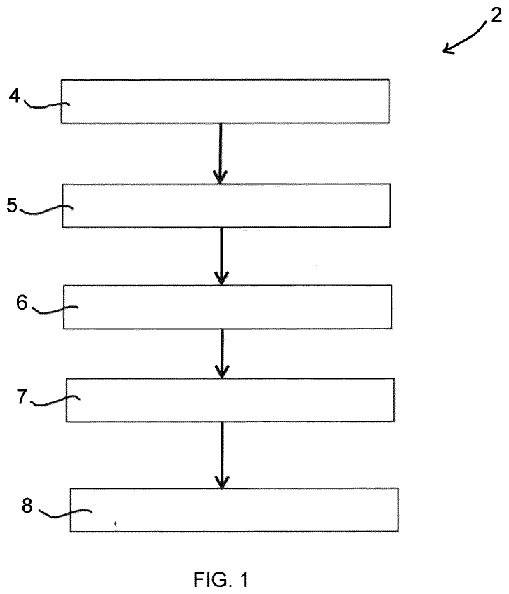

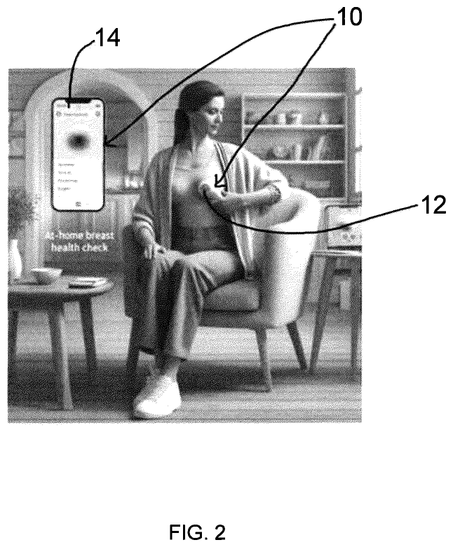

With reference to FIG. 1 of the drawings, a detection method in accordance with a first aspect of the invention is designated generally by the reference numeral 2 and includes:
as a first step 4 providing at least two piezoelectric sensors spaced a predetermined distance apart from one another;
as a second step 5 providing a plastically deformable material and locating said at least two piezoelectric sensors on the plastically deformable material;
as a third step 6 providing a rigid material adjacent said at least two piezoelectric sensors;
as a fourth step 7 compressing said at least two piezoelectric sensors between the rigid material and a material to be measured, thereby to produce a voltage signal in said at least two piezoelectric sensors; and as a fifth step 8 transforming the voltage signals from said at least two piezoelectric sensors into a measurement of a desired property of the material being measured.

One or more examples of the method will be explained hereinbelow with reference to a portable piezoelectric detection system, in accordance with a second aspect of the invention, designated generally by the reference numeral 10.

The portable piezoelectric detection system 10 is configured for detecting breast cancer and includes a detection device 12; and a mobile application which can be installed, in use, on a smartphone 14 or cellular phone 14 and which is operable for displaying information including visual data provided for the user; and an electrically coupled system 22.

The detection device 12 includes an array of piezoelectric sensors 16, a plastically deformable material layer 18, a rigid layer 20, and an electrically coupled system for processing signals (in the form of voltages) received from the sensors 16, as will be explained in more detail hereinbelow.

The array of piezoelectric sensors 16 are arranged a predetermined distance apart from one another and an equal distance apart from one another. The array of piezoelectric sensors 16 are imbedded in the plastically deformable layer 18.

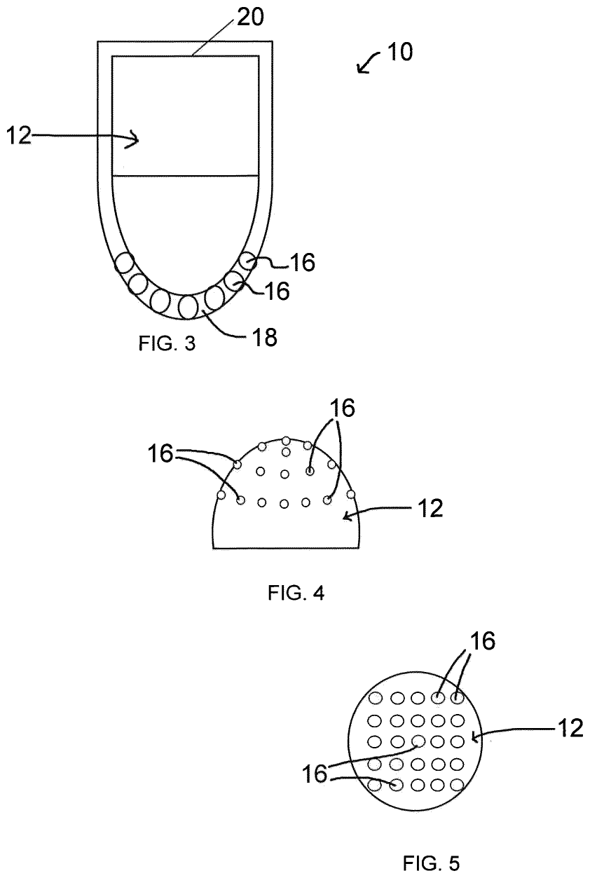

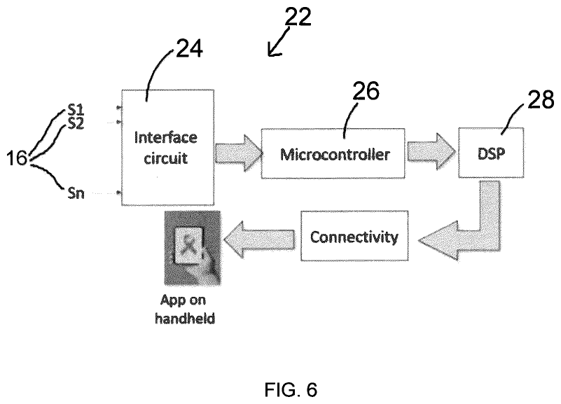

The rigid layer 20 is located adjacent to and underneath the plastically deformable material layer 18 and has hemispherical shape, as shown in FIG. 3 of the drawings. More particularly, the sensors 16 are embedded within the plastically deformable layer which is composed of a polymer soft material, forming a flexible yet stable matrix that can conform to various surfaces, such as the external breast surface.

The piezoelectric sensors 16 are strategically distributed across the hemispherical surface defined by the rigid layer 20 and designed to detect minute changes in pressure across its sensors 16 without the need for external electrical biasing or control signals. This enables the detection of mechanical stress generated by tissue anomalies or changes.

In use, the sensors 16 each produce a voltage in response to a force applied to the piezoelectric sensors 16, when the piezoelectric sensors 16 are compressed, in use, between a material to be measured and the rigid layer 20.

More specifically, the material to be measured is in the form of living tissue, specifically, but not exclusively breast tissue.

In use, the user will hold the device 12 in her hand an excerpt a force on the device 12 to trap the piezoelectric sensors 16 between the breast tissue to be measured and the rigid layer 20, thereby to produce a voltage which constitutes a signal which the electrically coupled system processes. In use, the sensors 16 monitor mechanical stresses and mechanical changes in breast tissue, as will be explained in more detail hereinbelow.

The inventor has found that it is important to select appropriate piezoelectric materials for the sensors 16 such that the sensors 16 are sensitive and stable enough to detect the subtle mechanical stresses associated with early-stage breast cancer through changes in tissue stiffness or other related biomarkers.

The inventor has found that it is important to design the array of piezoelectric sensors such that the array can be comfortably worn on the breast area. The array should cover a sufficient surface area to ensure comprehensive monitoring across the entire breast.

The inventor envisages that it is possible to integrate the array of sensors 16 into a wearable device, such as a specialized bra or patch, that maintains sensor contact with the skin without significant discomfort. It is also important to ensure the device is adjustable to accommodate different body sizes and shapes.

The electrically coupled system 22 includes electric circuits connected to the sensors 16 and configured to process the signals. The electric circuits include an interface circuit 24, a microcontroller 26, a digital signal processor (DSP) 28; a communication module and a storage module (not shown).

The interface circuit 24 is configured for receiving and amplifying the signals received from each sensor 16. More particularly, the interface circuit 24 is configured to format data for processing by the microcontroller 26, as will be explained in more detail hereinbelow.

The microcontroller 26 is configured for receiving amplified signals from the interface circuit 24. The DSP 28 is configured for processing complex computations on digital signals received from the microcontroller 26. For example, the DSP 28 is operable for analysing patterns or detecting anomalies, as explained below.

The communication module is configured for communication with the portable mobile device, such as, for example, the cellular phone 14 or smartphone 14.

When pressure is applied, in use, either manually or due to changes in the breast tissue itself, the piezoelectric sensors 16 generate electrical signals in response to the mechanical stress. These signals are then collected and initially processed by the interface circuit 24. The interface circuit 24 acts as a critical bridge, funneling the raw sensor data into the microcontroller 26, which serves as the system's central processor. The microcontroller 28 interprets the sensor data, which is subsequently relayed to the Digital Signal Processor (DSP) 28. The DSP 28 performs complex computations on these digital signals to analyze patterns and detect any anomalies that might indicate tissue changes indicative of health issues.

A significant feature of this system 10 is its connectivity component which is facilitated by the communication module and which allows the system 10 to communicate with external devices, such as the smartphone 14. This capability enables a user-friendly application to display the interpreted data visually, making it accessible and easier for users to understand.

The system 10 is designed to provide a detailed map of the pressure distribution over the specific area of the breast being examined. This pressure map is generated and compared against baseline maps collected from several healthy subjects to identify deviations that could indicate abnormalities.

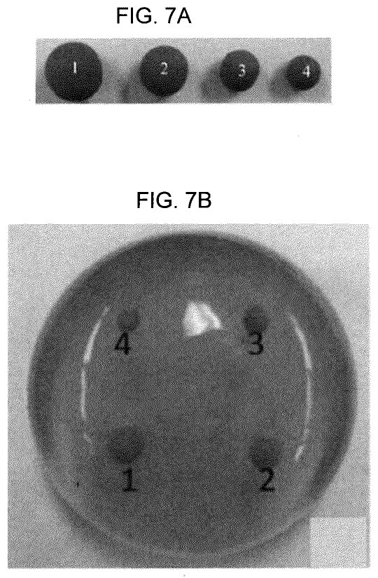

In use, the system 10 will be employed to identify tumors imbedded within a breast model, as illustrated in FIG. 7B of the drawings. FIG. 7A shows a variety of tumors of different sizes, while FIG. 7B shows the various tumors of FIG. 7A imbedded within a gelatin-based breast model.

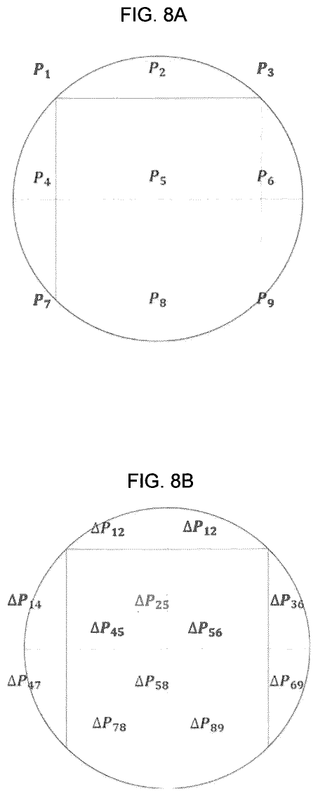

In use, the mapping shown in FIGS. 8A and 8B is derived directly from sensory output from the sensors 16. More specifically voltage signals received from the sensors 16 will be utilized to determine the corresponding velocity and dimensions of the tissue as well as location and/or size of tumors in the tissue as explained below.

### Example 1-Generation of Images

The various sensors 16 each produce a voltage signal which can be transformed into a desired property of the material being measured. For example, the voltage signals at each sensor 16 can be transformed mathematically into pressure readings as shown in FIG. 8A of the drawings. More specifically, the voltage signal of each sensor 16 can be mathematically transformed into pressure readings designated P₁ through P₉ in FIG. 8A of the drawings.

The pressure readings P₁ through P₉ are then used to calculate pressure values ΔP₁₂, ΔP₁₂, ΔP₁₄, ΔP₄₅, ΔP₂₅, ΔP₅₆, ΔP₃₆, ΔP₄₇, ΔP₇₈, ΔP₅₈, ΔP₈₉, ΔP₆₉, as shown in FIG. 8B of the drawings.

Mathematical modeling and/or graphical data representation is then applied to the data shown in FIG. 8B to produce the graphic output of FIG. 10.

The inventor envisages that various graphic outputs in the form of the graphic output of FIG. 10 can be obtained for a number of different reference subjects. Each reference subject can be labeled as “free of tumors” or as “having tumors”. As such, the inventor envisages that a reference library comprising graphic outputs of patients with tumors and patients without tumors can be constructed.

In use, voltage signals from the sensors 16 applied to breast tissue of a user can be used to construct graphic output in the form disclosed in FIG. 10 and this graphic output can then be compared (by the DSP 28) to the graphic output in the reference library in order to determine whether the graphic output of the user corresponds with graphic output of users with or without tumors. In use, the reference library may be remotely located on the cloud, or alternatively may be stored on the storage module. Advantageously, the piezoelectric detection system 10 can therefore classify the breast tissue of the user as likely to be with tumour or without tumour.

The applicant envisages that the voltage signals at each sensor can alternatively, or additionally be used to transform the voltage signal into other properties of the breast tissue being measured, such as, for example, viscosity of the tissue, radius of a blood vessel of the tissue, reaction forces of the tissue, etc.

In this way, the detection device 10 is therefore operable for detecting material properties of the breast tissue, as explained above.

If abnormalities are detected, the system can signal the need for further clinical investigations, facilitating early intervention and more detailed examinations if necessary.

### Example 2-Generating Images from the Corresponding Piezoelectric Measurements and Physiological Properties of Tissue

Piezoelectric materials possess the capacity to generate an internal electric field when exposed to mechanical stress. The relationship between the open circuit output voltage (V_{OC}) of a piezoelectric sensor 18 with a thickness (T) and the mechanical stress (σ) applied can be described by the piezoelectric voltage constant (g):

$${V}_{oc} = g σ T \qquad ( 1 )$$

In the present method, this mechanical stress is generated by the movement of blood within the vessel, and it can be estimated as:

$$σ = \frac{4 η Q}{π {r}^{3}} \qquad ( 2 )$$

Here, η represents the viscosity of the blood, Q denotes the blood flow rate within the vessel, and r signifies the inner radius of the vessel. Blood viscosity and the piezoelectric constant are established parameters. The viscosity, which quantifies the thickness of the fluid, can be assessed for an individual through conventional, pre-existing techniques.

To calculate the flow rate, it's essential to initially establish the inner radius. The flow rate can be expressed as the product of the cross-sectional area of the vessel (A=πr2) and the fluid velocity (ν) at a specific location, as follows:

$$Q = Av = π r 2 v \qquad ( 3 )$$

When equation (3) is inserted into equation (2), r can be represented as follows:

$$r = \frac{4 η v}{σ} \qquad ( 4 )$$

The fluid velocity can be derived by employing two neighboring piezoelectric sensors positioned as illustrated in FIG. 1 at a separation distance (d) and assessing the phase shift or time delay (Δt) between their respective output signals. Therefore, ν can be calculated by dividing the travel distance by the time delay, expressed as follows mathematically:

$$v = d / Δ t \qquad ( 5 )$$

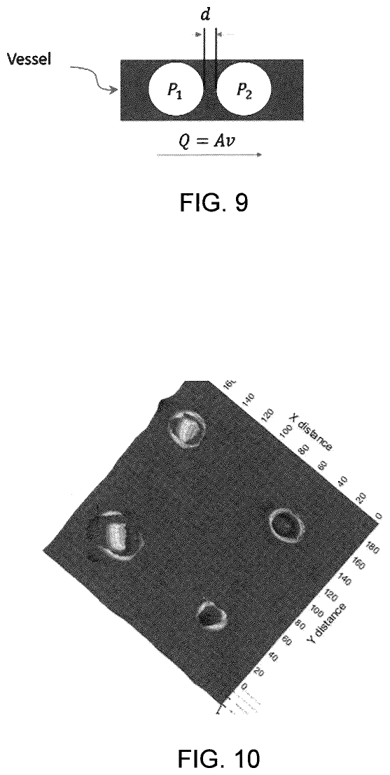

FIG. 9 illustrates two identical piezoelectric sensors 16.

The distance “d” between the two sensors 16 is considered to be equal to the distance between the centers of the circles or between the two edges.

Equation (2) is a well-established formula that describes the stress induced in a blood vessel due to the propagation of blood flow. In this equation, the symbol “o” represents the stress. Equation (2) serves as the basis for deriving the radius (r) of the blood vessel. In our work, “o” is the value measured using a piezoelectric sensor. The viscosity (n) is a parameter that can be determined using standard measurement tools. The key aspect of current approach is to measure “o” and the velocity; with a predetermined value for viscosity, the inventor can estimate the various parameters of the blood vessels and the breast tissue.

The proposed procedure is detailed in the following steps:

Step 1: The time delay was determined by monitoring the shift between the two signals across multiple cycles and calculating the average value based on these observations.

Step 2: The spacing between the two identical piezoelectric sensors should be set to the possible minimum, initially to be set at 1 mm.

Step 3: The corresponding velocity was determined by dividing the distance by the time (I/At), with the velocity expressed in meters per second.

Step 4: The pressure is then determined by rearranging equation (1) to solve for σ=V_{OC}/gT.

Step 5: inserting equation (4) in (3), yields

$$Q = \frac{16 {πη}^{2} {v}^{3}}{{σ}_{d}^{2}}$$

where σd=σ2−σ1.

Step 6: compute r using

$$r = \frac{4 η v}{{σ}_{d}} .$$

Step 7: Relocate the sensory system to a different position and recompute the mentioned parameters values at this new location by following the previously outlined steps.

Step 8: Map these parameters spatially and create the corresponding image.

Step 9: Analysis the image for possible abnormalities.

The developed probe will be employed to identify tumors embedded within a breast model, as illustrated in FIG. 7 of the drawings. FIG. 7(a) shows a variety of tumors of different sizes, and FIG. 7(b) demonstrates these tumors embedded within a gelatin-based breast model.

The inventor believes it is advantageous to develop a miniaturized electronic system for data acquisition that collects outputs from the piezoelectric sensors 16. This system includes amplification, filtering, and digital conversion capabilities. The inventor has found that it is advantageous to use signal processing algorithms to analyze the sensor outputs, focusing on detecting patterns or changes indicative of tissue anomalies.

The inventor has found that it is necessary to calibrate the system 10 using known standards to ensure accurate readings of mechanical stress. It is further necessary to adjust the calibration to account for individual variability in breast tissue density and other physiological factors.

The inventor has found that it is necessary before deployment, to validate the system's sensitivity and specificity for cancer detection against clinically obtained data, such as mammograms or MRI scans, to ensure it provides reliable and accurate results.

The inventor has found that it is advantageous to design a user-friendly interface for the device that allows users to operate the system easily and view their results and to include clear instructions and safety guidelines.

Advantageously the system includes implementation of data logging and possibly real-time data transmission to healthcare providers. It is important to ensure privacy and security in data handling, adhering to medical data regulations.

The inventor has found that whilst the invention has been described with reference to detection of breast cancer, the device 12 and system 10 may equally be used for any other types of detailed surface monitoring, either in the medical field or in other fields.

The inventor has found that the system and device are particularly advantageous as they provide a private, inexpensive, non-intrusive, comfortable and convenient means of breast cancer screening which can be performed in the comfort and privacy of the home. As such, the inventor envisages that the user will utilize the device and system to screen for breast cancer more often. This will no-doubt lead to early detection of early changes in the tissue which may signal warning signs which will cause the user to seek medical screening and further investigation. As such, early detection of cancer will result in much better treatment outcomes for the patient.

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention. For example, the system 10 may be operable to measure viscosity of the tissue, or any other physical characteristics of the tissue. Such measured characteristics can likewise be compared to characteristics of a reference library.

1. A detection method for detecting material properties of breast tissue includes:
providing at least two piezoelectric sensors spaced a predetermined distance apart from one another and operable for measuring pressure;
providing a plastically deformable material forming a flexible, yet stable matrix which can conform to a shape of an external breast surface, and locating said at least two piezoelectric sensors on the plastically deformable material for maintaining sensory contact with the skin without significant discomfort;
providing a rigid material adjacent said at least two piezoelectric sensors;
compressing said at least two piezoelectric sensors between the rigid material and a material to be measured, thereby to produce a voltage signal in said at least two piezoelectric sensors; and
transforming the voltage signals from said at least two piezoelectric sensors into a measurement of a desired property of the material being measured.

2. The detection method as claimed in claim 1, wherein the material to be measured is in a form of living tissue.

3. The detection method as claimed in claim 2, wherein the living tissue is in the form of breast tissue.

4. The detection method as claimed in claim 3, wherein the voltage signals from said at least two piezoelectric sensors is transformed into a measurement of one or more selected from a group comprising: viscosity of blood flowing in the breast tissue beneath the sensors, reaction forces of the breast tissue, radius of a blood vessel of the breast tissue, velocity of blood flow in a blood vessel of the breast tissue.

5. A detection device for detecting material properties of breast tissue including:
at least two piezoelectric sensors spaced a predetermined distance apart from one another and operable for measuring pressure;
a plastically deformable material layer forming a flexible, yet stable matrix which can conform to a shape of an external breast surface;
said at least two piezoelectric sensors being located adjacent the plastically deformable material layer for maintaining sensory contact with the skin without significant discomfort; and
a rigid layer located adjacent the plastically deformable material layer, in an arrangement wherein, in use, said at least two piezoelectric sensors each produce a voltage signal in response to a force applied to said at least two piezoelectric sensors when said at least two piezoelectric sensors are compressed, in use, between a material to be measured and the rigid layer.

6. The detection device as claimed in claim 5, wherein the at least two piezoelectric sensors comprise a plurality of sensors.

7. The detection device as claimed in claim 6, wherein the plurality of sensors comprises an array of sensors arranged a predetermined distance apart from one another.

8. The detection device as claimed in claim 7, wherein said predetermined distance is a uniform distance such that the array of sensors are spaced an equal distance apart from one another.

9. The detection device as claimed in claim 5, wherein the material to be measured is in a form of living tissue.

10. The detection device as claimed in claim 9, wherein the living tissue is in the form of breast tissue.

11. The detection device as claimed in claim 6, wherein the device is operable for transforming the voltage signal from the plurality of sensors into pressure readings for detecting changes in pressure between adjacent sensors in the plurality of sensors.

12. The detection device as claimed in claim 11, wherein the material to be measured is in a form of breast tissue and wherein the device is operable to construct a map of the pressure distribution for specific areas of the breast.

13. The detection device as claimed in claim 12, wherein the device is operable to compare the map with one or more maps obtained from a reference library.

14. The detection device as claimed in claim 13, wherein said reference library includes one or more maps of pressure distributions obtained from healthy individuals using the device.

15. The detection device as claimed in claim 5, wherein said at least two piezoelectric sensors are at least partially imbedded in the plastically deformable layer.

16. The detection device as claimed in claim 10, wherein the material properties are one or more of the following: a mechanical stress of the breast tissue, a viscosity of the tissue, a radius of a blood vessel of the tissue, a reaction force of the tissue.

17. The detection device as claimed in claim 5, wherein the rigid layer has a hemispherical shape.

18. A portable piezoelectric detection system, the system including: a device as claimed in claim 5; and
a mobile application which can be installed, in use, on a smartphone or cellular phone and which is operable for displaying information received from the device.
